SEQUENCE LISTING (6 sequences)

SEQ ID NO: 1 - protein, 383 aa, Salmonella typhimurium
MAEESDDDKTEAPTPHRLEKAREEGQIPRSRELTSLLILLVGVCIIWFGGESLARQLAGMLSAGLHFDHRMVNDPNLILGQIILLIKAAMMALLPLIAGVVLVALISPVMLGGLIFSGKSLQPKFSKLNPLPGIKRMFSAQTGAELLKAVLKSTLVGCVTGFYLWHHWPQMMRLMAESPIVAMGNALDLVGLCALLVVLGVIPMVGFDVFFQIFSHLKKLRMSRQDIRDEFKESEGDPHVKGKIRQMQRAAAQRRMMEDVPKADVIVTNPTHYSVALQYDENKMSAPKVVAKGAGLIALRIREIGAEHRVPTLEAPPLARALYRHAEIGQQIPGQLYAAVAEVLAWVWQLKRWRLAGGQRPPQPENLPVPEALDFMNEKNTDG

SEQ ID NO: 2 - protein, 350 aa, Aquifex aeolicus
MAEEHKTERATPYKRRKVREEGNVAKSHEIASSLVVLLSLLLLLFLGTYIAKEVILIFLAVTGYVHADISELGSLYENFYENIVKVLTPLFFLALLVVILSHVAQFGFIFTLKPLSFKWERINPFEGIKRLISLTTLFETVKNTLKAFLLIGIAVFVLKGSLYFFLSSSTYPLAETLKSFIKTSAITLITLGVVALLIAFLDYAFKRWQYEKKIMMSRRELKEEYKQLEGHPEVKSRIKARMRELAKSRMMAEVPKATVVITNPTHIAIALKYNPEKDKAPVVVAKGKGTIAQKIVEIAENYSIPVVRKPELARALYPAVEVGKEISPKFYKAVAEIIAYVMFKKKKVYA

SEQ ID NO: 3 - protein, 345 aa, Escherichia coli
MSEKTEKPTPKKLRDLKKKGDVTKSEEVMAAVQSLILFSFFSLYGMSFFVDIVGLVNTTIDSLNRPFLYAIREILGAVLNIFLLYILPISLIVFVGTVTTGVSQIGFIFAVEKIKPSAQKISVKNNLKNIFSVKSIFELLKSVFKLVIIVLIFYFMGHSYANEFANFTGLNAYQALVVVAFFVFLLWKGVLFGYLLFSVFDFWFQKHEGLKKMKMSKDEVKREAKDTDGNPEIKGERRRLHSEIQSGSLANNIKKSTVIVKNPTHIAICLYYKLGETPLPLVIETGKDAKALQIIKLAELYDIPVIEDIPLARTLYKNIHKGQYITEDFFEPVAQLIRIAIDLDY

SEQ ID NO: 4 - protein, 354 aa, Yersinia pestis
MSGEKTEQPTPKKIRDARKKGQVAKSKEVVSTALIVALSAMLMGLSDYYFEHFSKLMLIPAEQSYLPFSQALSYVVDNVLLEFFYLCFPLLTVAALMAIASHVVQYGFLISGEAIKPDIKKINPIEGAKRIFSIKSLVEFLKSILKVVLLSILIWIIIKGNLVTLLQLPTCGIECITPLLGQILRQLMVICTVGFVVISIADYAFEYYQYIKELKMSKDEIKREYKEMEGSPEIKSKRRQFHQEIQSRNMRENVKRSSVVVANPTHIAIGILYKRGETPLPLVTFKYTDAQVQTVRKIAEEEGVPILQRIPLARALYWDALVDHYIPAEQIEATAEVLRWLERQNIEKQHSEML

SEQ ID NO: 5 - protein, 356 aa, Salmonella typhimurium
MSSNKTEKPTKKRLEDSAKKGQSFKSKDLIIACLTLGGIAYLVSYGSFNEFMGIIKIIIADNFDQSMADYSLAVFGIGLKYLIPFMLLCLVCSALPALLQAGFVLATEALKPNLSALNPVEGAKKLFSMRTVKDTVKTLLYLSSFVVAAIICWKKYKVEIFSQLNGNIVGIAVIWRELLLALVLTCLACALIVLLLDAIAEYFLTMKDMKMDKEEVKREMKEQEGNPEVKSKRREVHMEILSEQVKSDIENSRLIVANPTHITIGIYFKPELMPIPMISVYETNQRALAVRAYAEKVGVPVIVDIKLARSLFKTHRRYDLVSLEEIDEVLRLLVWLEEVENAGKDVIQPQENEVRH

SEQ ID NO: 6 - protein, 342 aa, Shigella flexneri
MANKTEKPTPKKLKDAAKKGQSFKFKDLTTVVIILVGTFTIISFFSLSDVMLLYRYVIINDFEINEGKYFFAVVIVFFKIIGFPLFFCVLSAVLPTLVQTKFVLATKAIKIDFSVLNPVKGLKKIFSIKTIKEFFKSILLLIILALTTYFFWINDRKIIFSQVFSSVDGLYLIWGGLFKDIILFFLAFSIFVIILDFVIEFILYMKDMMMDKQEIKREYIEQEGHFETKSRRRELHIEILSEQTKSDIRNSKLVVMNPTHIAIGIYFNPEIAPAPFISLIETNQCALAVRKYANEVGIPTVRDVKLARKLYKTHTKYSFVDFEHLDEVLRLIVWLEQVENTH

(19) United States
(12) Patent Application Publication
(10) Pub. No.: US 2017/0049779 A1
(43) Pub. Date: Feb. 23, 2017

(54) FLAGELLAR AND NEEDLE COMPLEX (INJECTOSOME) LOOP AS ANTI BACTERIAL DRUG TARGET

(71) Applicant: OKINAWA INSTITUTE OF SCIENCE AND TECHNOLOGY SCHOOL CORPORATION, Kunigami-gun (JP)

(72) Inventors: Fadel Alexis SAMATEY, Kunigami-gun (JP); Vladimir A. MESHCHERYAKOV, Kunigami-gun (JP); Hideyuki MATSUNAMI, Kunigami-gun (JP); Akio KITAO, Tokyo (JP)

(73) Assignee: OKINAWA INSTITUTE OF SCIENCE AND TECHNOLOGY SCHOOL CORPORATION, Kunigami-gun (JP)

(21) Appl. No.: 15/237,244

(22) Filed: Aug. 15, 2016

Related U.S. Application Data
(62) Division of application No. 14/770,708, filed on Aug. 26, 2015.
(60) Provisional application No. 61/770,046, filed on Feb. 27, 2013.

(51) Int. Cl. A61K 31/522; A61K 31/505

(52) U.S. Cl. CPC A61K 31/522; A61K 31/505

(57) ABSTRACT

The present invention relates to a method for screening a compound that inhibits secretion of toxins into host-cell cytoplasm by virulent bacteria using a needle type III secretion system. The compound of the invention is selected by screening for a compound which interacts with a loop region of the cytoplasmic domain of the membrane protein FlhB from Salmonella typhimurium or a paralog thereof Compositions including the compound of the invention, use of the compound, and methods of treating disorders caused by virulent bacteria are also provided.

This application is a Divisional of copending application Ser. No. 14/770,708, filed on Aug. 26, 2015, which was filed as PCT International Application No. PCT/JP2014/056082 on Feb. 27, 2014, which claims the benefit under 35 U.S.C. §119(e) to U.S. Provisional Application No. 61/770,046, filed on Feb. 27, 2013.

### REFERENCE TO SEQUENCE LISTING SUBMITTED VIA EFS-WEB

This application includes an electronically submitted sequence listing in .txt format. The .txt file contains a sequence listing entitled “Sequence_Listing_0283_0389PUS2.txt ” created on Aug. 5, 2016, and is 20,480 bytes in size. The sequence listing contained in this .txt file is part of the specification and is hereby incorporated by reference herein in its entirety.

### FIELD OF THE INVENTION

The present invention relates to the type III secretion system of virulent bacteria, and more specifically, a method for screening a compound that inhibits secretion of toxins into host-cell cytoplasm by virulent bacteria using a needle type III secretion system.

### BACKGROUND OF THE INVENTION

The bacterial flagellum is a large, complex structure that is used by many bacteria as a motility organelle. It consists of three main substructures: the basal body, the hook and the filament. Most of the flagellar proteins are initially localized inside of the cell and translocated across the cell membrane by the flagellum-specific secretion apparatus that is evolutionarily and structurally related to the virulence type III secretion system (Non-Patent Literature 1 and 2). Protein export by the flagellar type III secretion system is highly regulated. The secretion system first exports rod/hook type proteins until the hook reaches an appropriate length. After that the secretion system switches substrate specificity from hook-type export to filament-type export (Non-Patent Literature 3 and 4). In Salmonella, the substrate-specificity switch is controlled by two proteins: FliK and FlhB (Non-Patent Literature 5-7).

FlhB is an essential membrane protein of the flagellar type III secretion system. It consists of two domains: a hydrophobic N-terminal part (FlhB_{TM}) that is predicted to have four transmembrane helices, and a C-terminal cytoplasmic domain (FlhB_{C}) (Non-Patent Literature 8). The two domains are connected by a flexible linker. This linker is a highly conserved part of the FlhB protein and is essential for the type III secretion system (TTSS). Deletions or point mutations in the linker region completely abort or significantly reduce secretion (Non-Patent Literature 9 and 10). The wild-type cytoplasmic domain of Salmonella FlhB undergoes autocatalytic cleavage between amino-acid residues Asn269 and Pro270 within a highly conserved NPTH sequence (Non-Patent Literature 11). This auto-cleavage is essential for the switching process (Non-Patent Literature 9 and 12). Mutation of Asn269 to Ala prevents cleavage and locks the export apparatus in the hook-type specificity state.

FlhB_{C} has been shown to interact with several soluble components of the TTSS: FliH, FliI, FliJ (Non-Patent Literature 13), the cytoplasmic part of membrane protein FlhA (Non-Patent Literature 14) and the hook-length control protein FliK (Non-Patent Literature 15 and 16). Interaction of FlhB with FliK is suggested to be important for the substrate-specificity switching process (Non-Patent Literature 17). Cells with a deleted fliK gene produce an abnormally long hook, termed a “polyhook”, without any filament attached (Non-Patent Literature 6).

### PRIOR ART LITERATURES

### Non-Patent Documents

[Non-Patent Literature 1] Aizawa, S.-I. (2001). FEMS Microbiol. Lett. 202, 157-164. Ashkenazy, H., Erez, E., Martz, E., Pupko, T., Ben-Tal, N. (2010). Nucleic Acids Res. 38, W529-W533.
[Non-Patent Literature 2] Blocker, A., Komoriya, K., Aizawa, S.-I. (2003). Proc. Natl. Acad. Sci. USA 100, 3027-3030.
[Non-Patent Literature 3] Minamino, T. and Macnab, R. M. (1999). J. Bacteriol. 181, 1388-1394.
[Non-Patent Literature 4] Hirano, T., Minamino, T., Namba, K., Macnab, R. M. (2003). J. Bacteriol. 185, 2485-2492.
[Non-Patent Literature 5] Kutsukake, K., Minamino, T., Yokoseki, T. (1994). J. Bacteriol. 176, 7625-7629.
[Non-Patent Literature 6] Hirano, T., Yamaguchi, S., Oosawa, K., Aizawa, S.-I. (1994). J. Bacteriol. 176, 5439-5449.
[Non-Patent Literature 7] Williams, A., Yamaguchi, S., Togashi, F., Aizawa, Kawagishi, I., Macnab, R. M. (1996). J. Bacteriol. 178, 2960-2970.
[Non-Patent Literature 8] Minamino, T., Iino, T., Kutsukake, K. (1994). J. Bacteriol. 176, 7630-7637.
[Non-Patent Literature 9] Fraser, G. M., Hirano, T., Ferris, H. U., Devgan, L. L., Kihara, M., Macnab, R. M. (2003). Mol. Microbiol. 48, 1043-1057.
[Non-Patent Literature 10] Zarivach, R., Deng, W., Vuckovic, M., Felise, H. B., Nguyen, H. V., Miller, S. I., Finlay, B. B., Strynadka, N. C. J. (2008). Nature, 453, 124-127.
[Non-Patent Literature 11] Minamino, T., and Macnab, R. M. (2000a). J. Bacteriol. 182, 4906-4914.
[Non-Patent Literature 12] Ferris, H. U., Furukawa, Y., Minamino, T., Kroetz, M. B., Kihara, M., Namba, K., Macnab, R. M. (2005). J. Biol. Chem. 280, 41236-41242.
[Non-Patent Literature 13] Minamino, T., and Macnab, R. M. (2000b). Mol. Microbiol. 35, 1052-1064.
[Non-Patent Literature 14] Zhu, K., Gonzales-Pedrajo, B., Macnab, R. M. (2002). Biochemistry, 41, 9516-9524.
[Non-Patent Literature 15] Minamino, T., Saijo-Hamano, Y., Furukawa, Y., Gonzales-Pedrajo, B., Macnab, R. M., Namba, K. (2004). J. Mol. Biol. 341, 491-502.
[Non-Patent Literature 16] Morris, D. P., Roush, E. D., Thompson, J. W., Moseley, M. A., Murphy, J. W., McMurry, J. L. (2010). Biochemistry, 49, 6386-6393.
[Non-Patent Literature 17] Ferris, H. U., Minamino, T. (2006). Trends Microbiol. 14, 519-526.
[Non-Patent Literature 18] Deane, J. E., Graham, S. C., Mitchell, E. P., Flot, D., Johnson, S., Lea, S. M. (2008). Mol. Microbiol. 69, 267-276.
[Non-Patent Literature 19] Wiesand, U., Sorg, I., Amstutz, M., Wagner, S., van den Heuvel, J., Luhrs, T., Cornelis, G. R., Heinz, D. W. (2009). J. Mol. Biol. 385, 854-866.
[Non-Patent Literature 20] Lountos, G. T., Austin, B. P., Nallamsetty, S., Waugh, D. S. (2009). Protein Sci. 18, 467-474.
[Non-Patent Literature 21] Mizuno, S., Amida, H., Kobayashi, N., Aizawa, S.-I., Tate, S.-I. (2011). J. Mol. Biol. 409, 558-573.
[Non-Patent Literature 22] Veenendaal A. K. J, Sundin C., Blocker A. J. (2009). J. Bacteriol. 191(2) 563-570.

### OBJECT OF THE INVENTION

Several structures of the cytoplasmic domain of FlhB paralogs from the needle TTSS have been published (Non-Patent Literature 10, and 18-20). However, no structural information about FlhB from the flagellar secretion system is available. Thus, it is an object of the present invention to clarify crystal structures of the cytoplasmic domain of flagellar FlhB from two organisms: Salmonella typhimurium and Aquifex aeolicus.

Based on the structural relationship between FlhB_{c} of the flagellar secretion system and that of the needle type III secretion system utilized by a number of virulent bacteria for the secretion of toxins into the host-cell cytoplasm, it is also an object of the present invention to provide a method for identifying compounds that inhibits secretion of toxins into host-cell cytoplasm by virulent bacteria using the structural information of FlhBc.

### SUMMARY OF THE INVENTION

The most important findings of the present invention is that flexibility of the large non-conserved loop in the globular domain of FlhB_{C} is necessary for function of the whole secretion system. Deletion of the loop or its mutation to less flexible proline residues makes FlhB_{C} more rigid and thus aborts or significantly reduces secretion. Taking into account similarity between the flagellar and needle proteins, this loop could be a promising target for creation of novel drugs against pathogenic bacteria, and the following inventions have been completed.

In one aspect of the present invention, there is provided a method for screening a compound that inhibits secretion of toxins into host-cell cytoplasm by virulent bacteria using a needle type III secretion system, the method comprising the steps of:

contacting a candidate compound with a C-terminal cytoplasmic domain of a membrane protein FlhB (FlhB_{C}) from Salmonella typhimurium or a paralog thereof,

analyzing interaction of the candidate compound with or around a loop region of the cytoplasmic domain, and

selecting a compound that reduces flexibility of the loop region or a linker that connects the transmembrane and cytoplasmic domains of FlhB or the paralog thereof,

wherein the selected compound is indicated to inhibit the secretion of toxins by virulent bacteria.

In preferred embodiments, the virulent bacteria in the method of the invention is selected from the group consisting of Salmonella typhimurium, Aquifex aeolicus, Yersinia pestis, Shigella flexneri, enterohemorrhagic Escherichia coli, Pseudomonas aeruginosa, and Vibrio parahaemolyticus.

In particular embodiments, the paralog of the membrane protein FlhB from Salmonella typhimurium is EscU from Escherichia coli, YscU from Yersinia pestis, SpaS from Salmonella typhimurium, and Spa40 from Shigella flexneri.

In other particular embodiments, the loop region of the cytoplasmic domain of FlhB or the paralog thereof in the method of the invention consists of the amino acid residues ENKMS₂₈₁₋₂₈₅ in Salmonella numeration.

In other particular embodiments, the compound that inhibits the secretion of toxins by virulent bacteria in the method of the invention is capable of binding to the loop region of the cytoplasmic domain of FlhB or the paralog thereof, or a flanking region thereof,

wherein the flanking region comprising a conserved amino acid residue Tyr279 and Pro287 in Salmonella numeration.

In other particular embodiments, the interaction of the compound with or around the loop region of the cytoplasmic domain of FlhB or the paralog thereof in the method of the invention is determined whether or not the compound differentially binds to the membrane protein FlhB from Salmonella typhimurium and its Δ(281-285) mutant protein.

In other particular embodiments, the compound that inhibits the secretion of toxins by virulent bacteria in the method of the invention is an antibody or a fragment thereof, an aptamer or a small molecular compound.

In another aspect of the present invention, there is provided a method for screening a compound that inhibits secretion of toxins into host-cell cytoplasm by virulent bacteria using a needle type III secretion system, the method comprising the steps of:
i) selecting a candidate compound capable of interacting with a membrane protein FlhB from Salmonella typhimurium or a paralog thereof, by hydrogen bond between a loop region of the cytoplasmic domain of the FlhB or the paralog thereof and the candidate compound,
ii) contacting the candidate compound with bacteria that has flagellar and needle type III secretion system, and
iii) selecting a compound that reduces secretion of proteins from the bacteria and/or motility of the bacteria using the flagellar,
   wherein the selected compound is indicated to inhibit the secretion of toxins by virulent bacteria.

In one embodiment, the hydrogen bond between a loop region of the cytoplasmic domain of the membrane protein of FlhB or the paralog thereof and the candidate compound in the method of the invention is formed via at least one side chain of the amino acid residues ENKMS₂₈₁₋₂₈₅ in Salmonella numeration.

In another aspect of the present invention, there is provided a compound identified by the method of the invention.

In another aspect of the present invention, there is provided a pharmaceutical composition for inhibiting secretion of toxins into the host-cell cytoplasm, comprising the identified compound of the invention, or a pharmaceutically acceptable salt thereof.

In one embodiment, the compound of the pharmaceutical composition of the invention is selected from the group of:
7-(2,3-dihydroxypropyl)-1,3-dimethyl-3,7-dihydro-1H-purine-2,6-dione
5-(3,4,5-Trimethoxybenzy 1)pyrimidine-2,4-diamine
and pharmaceutically acceptable salt thereof.

In another aspect of the present invention, a method of treating disorders caused by virulent bacteria using a needle type III secretion system, the method comprising:

contacting the bacteria with the pharmaceutical composition of the invention, and

inhibiting the secretion of toxins by the virulent bacteria.

In another aspect of the present invention, there is provided a method for inhibiting secretion of toxins into the host-cell cytoplasm by virulent bacteria using a needle type III secretion system, the method comprising:

contacting the bacteria with the pharmaceutical composition of the invention, and

inhibiting the secretion of toxins by the virulent bacteria.

In another aspect of the present invention, there is provided use of the composition of the invention for inhibiting secretion of toxins into the host-cell cytoplasm.

### BRIEF DESCRIPTION OF THE DRAWINGS

Figure Legends:

FIG. 1A: Molecular packing in the crystal of Salmonella FlhB_{C}. Asymmetric molecule and symmetry-related molecules are displayed in green and gray, respectively. Sodium ions are represented as magenta spheres, and zinc ions are shown as blue spheres.

FIG. 1B: Rotated enlarged view of the zinc-binding site (black box in FIG. 1A). The Fo-Fc electron density map is displayed in gray at a contour level of 5σ and was calculated without Zn atom.

FIG. 2A: Structure of flagellar FlhB_{C}. Ribbon representation of the crystal structure of Salmonella and Aquifex FlhB_{C}.

FIG. 2B: Electrostatic potential mapped on the surface of Salmonella FlhB_{C}. Electrostatics was calculated using APBS software (Baker, N. A., Sept, D., Joseph, S., Hoist, M. J., McCammon, J. A. (2001). Proc. Natl. Acad. Sci. USA 98, 10037-10041) and plotted at ±5 kT e-1.

FIG. 2C: Evolutionarily conserved residues of FlhB_{C}. The figure was prepared with ConSurf (http://consurf.tau.ac.il/) (Ashkenazy et al., 2010). Residues are colored accordingly to the conservation in amino acid sequences of 200 different FlhB proteins. Arrows mark position of the autocleavage site between β1 and β2.

FIG. 3A: Comparison of flagellar FlhB _{C} and its paralogs from needle type III secretion system. [[(a)]] Multiple sequence alignment of FlhB_{C} from S. typhimurium (Sal FlhB_{C}) (Swiiss-Prot. Accession number P40727) with FlhB _{C} from A. aeolicus (Aqu FlhB_{C}) (O67813), EscUC from E. coli (Q7DB59), YscUC from Yersinia pestis (P69986), SpaSC from S. typhimurium (P40702) and Spa40C from Shigella flexneri (Q6XVW1). Identical residues are boxed in red; similar residues are colored red. Alignment was done with Clustal W (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007). Bioinformatics, 23, 2947-2948; Goujon, M., McWilliam, H., Li, W., Valentin, F., Squizzato, S., Paern, J., Lopez, R. (2010). Nucleic Acids Res. 38, W695-699).

FIG. 3B: Stereo views of the superposition of Salmonella FlhB_{C} (blue) (PDB ID: 3B0Z), Aquifex FlhB_{C} (red) (PDB ID: 3B1S), EscUC (orange) (PDB ID: 3BZO), YscUC (gray) (PDB ID: 2JLI), SpaSC (green) (PDB ID: 3C01) and Spa40C (purple) (PDB ID: 2VT1).

FIG. 4A: Effect of mutations of the region ENKMS281-285 of Salmonella FlhB on the protein function. Ribbon representation of Salmonella FlhB_{C}; the region is in black.

FIG. 4B: Ability of FlhB variants with modified ENKMS region to complement a ΔflhB Salmonella strain MKM50. Motility was carried out on semi-solid agar plates at 303 K for 5 h. FlhB products were: 1) none, empty vector, 2) wild-type FlhB, 3) FlhB Δ(281-285), 4) FlhB AAAAA₂₈₁₋₂₈₅, and 5) FlhB PPPPP₂₈₁₋₂₈₅.

FIG. 4C: Immunoblotting using anti-FlgE and anti-FliC antibodies on the whole cell and culture supernatant fractions from MKM50 Salmonella strain producing different FlhB variants.

FIG. 5A: Flexibility of the N-terminal α-helix of Salmonella FlhB_{C} observed in MD simulation. Key residues and vectors used for MD analysis in Table 2. The vectors connecting residues 229-238, 238-256, 256-264, 265-269 and 238-264 are defined as V1-V5, respectively.

FIG. 5B: Structure variations of the N-terminal α-helix during MD when the globular domain is superimposed for the wild-type FlhB_{C}.

FIG. 5C: Structure variations of the N-terminal α-helix during MD when the globular domain is superimposed for FlhB _{C} Δ(281-285).

FIG. 5D: Structure variations of the N-terminal α-helix during MD when the globular domain is superimposed for FlhB_{C} AAAAA₂₈₁₋₂₈₅.

FIG. 5E: Structure variations of the N-terminal α-helix during MD when the globular domain is superimposed for FlhB_{C} PPPPP₂₈₁-₂₈₅. For each case, six snapshots were chosen based on the maximum and minimum values of D, θ14 and χ5 (see Table 2 for their definitions).

FIG. 6: Bacterial growth in presence of candidate compounds. The ordinate shows the optical density at 600 nm (OD 600). The abscissa shows the measured time points (2, 4, 6, 8 and 10 hours).

FIG. 7A: Inhibitory effect of screened compounds on FlgD secretion.

FIG. 7B: Western blots of suspension of Salmonella typhimurium strain SJW1103 incubated 8 hours with screened compounds.

### DETAILED DESCRIPTION OF THE INVENTION

Before the present invention is described in more detail below, it should be appreciated that the present invention is not limited to the particular methodology, protocols and reagents described herein as these may vary. It should be also appreciated that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

The term “FlhB_{C}” used herein refers to a C-terminal cytoplasmic domain of FlhB, which is an essential membrane protein of the flagellar type III secretion system. In the context of present invention, the FlhB from Salmonella typhimurium is preferred. FlhB from Salmonella typhimurium and FlhB _{C} thereof are described, e.g. in Non-Patent Literature 11. Typical example of an amino acid sequence of FlhB Salmonella typhimurium is provided in Swiss-Prot. Accession No. P40727 (SEQ ID NO: 1). For the purpose of present invention, the term “FlhB” also refers to a variant of FlhB from Salmonella typhimurium as long as the variant maintains its physiological activity and its crystalized property. The amino acid sequence of such variant may have an amino acid sequence at least 80%, 90% or 95% identical to SEQ ID NO: 1. The amino acid sequence of FlhB_{C} is easily determined from the FlhB defined above. Preferably, the amino acid sequence of FlhB _{C} is the amino acid position 219 to 383 of SEQ ID NO: 1 or variant thereof.

A paralog of FlhB from Salmonella typhimurium is also encompassed in the present invention. More specifically, the paralog include, but not limited to, FlhB from Aquifex aeolieus (a typical amino acid sequence is provided in Swiss-Prot. Accession No. O67813 (SEQ ID NO: 2)), EscU from Escherichia coli (a typical amino acid sequence is provided in (a typical amino acid sequence is provided in Swiss-Prot. Accession No. Q7DB59 (SEQ ID NO: 3)), YscU from Yersinia pestis (a typical amino acid sequence is provided in Swiss-Prot. Accession No. P69986 (SEQ ID NO: 4)), Spas from Salmonella typhimurium (a typical amino acid sequence is provided in Swiss-Prot. Accession No. P40702 (SEQ ID NO: 5)) and Spa40 from Shigella flexneri (a typical amino acid sequence is provided in Swiss-Prot. Accession No. Q6XVW1 (SEQ ID NO: 6)). Cytoplasmic domain of the paralog is easily determined from full length amino acid sequence of such paralogs.

The term “virulent bacteria” used herein refers to any bacterium which bears needle type III secretion system. Such bacteria can secrete toxins including AB toxin. The Example of virulent bacteria includes, but not limited to, Salmonella typhimurium, Aquifex aeolicus, Yersinia pestis, Shigella flexneri, enterohemorrhagic Escherichia coli, Pseudomonas aeruginosa, and Vibrio parahaemolyticus.

A loop region of FlhB_{C} or a paralog thereof of the present invention refers to the consecutive amino acid residues which constitute a long flexible loop connecting β2 and β3 strands in FlhB_{C} or a paralog thereof. The length of the loop is longer than necessary just for connecting two β strands. This loop region may be determined using structural information obtained from crystals of FlhBc or a paralog thereof. In this context, exemplary crystals are those crystalized from FlhB_{C} of Salmonella typhimurium, and from FlhBc Aquifex aeolicus which belongs P4₂2₁2 space group and C2 group, respectively. The crystal information of such crystals is shown in Table 1 and 2. Atomic coordinates and structure factors of structural information obtained from the above crystals are deposited in the PDB with accession codes 3BOZ and 3B IS for Salmonella typhimurium, and Aquifex aeolicus, respectively. The preferable loop region consists of the amino acid residues ENKM5₂₈₁-₂₈₅ in Salmonella numeration.

The loop region of FlhBc or a paralog thereof of the present invention can influences the flexibility of the N-terminal α-helix of FlhB_{C} or a paralog thereof. “Flexibility” of FlhB_{C} or a paralog thereof can be determined by any method known in the art. In one embodiment, flexibility of FlhB_{C} or a paralog thereof can be determined by Molecular Dynamic Simulation (MDS) using structural information of FlhBc or a paralog, as disclosed in the Examples herein below. In another embodiment, the change of flexibility of FlhB_{C} or a paralog thereof can be examined by the secretion assay or motility assay as disclosed in the Examples. In secretion assay, the reduction of the secretion activity of bacterium indicates the reduction of the flexibility of FlhB_{C}. Similarly in motility assay, the reduction of the motility activity of bacterium indicates the reduction of the flexibility of FlhB_{C}.

The term “flanking region” of the loop region used herein refers to the region comprises several amino acid sequence flanked to the N-terminal or C-terminal end of the loop region. The length of the flanking region may be 1 to 20, preferably 2 to 15, more preferably 5 to 10 amino acid length. Preferably, the flanking region comprises a conserved amino acid residue Tyr279 and Pro287 in Salmonella numeration.

As mentioned above, the present invention provides a method for screening a compound that inhibits secretion of toxins into host cell cytoplasm by virulent bacteria using a needle type III secretion system, the method comprising the step of:

contacting a candidate compound with a C-terminal cytoplasmic domain (FlhBc) of the membrane protein FlhB from Salmonella typhimurium or a paralog thereof,

analyzing interaction of the candidate compound with or around a loop region of the cytoplasmic domain (FlhBc), and

selecting a compound that reduces flexibility of the loop region, or a linker that connects the transmembrane and cytoplasmic domains of FlhB,

wherein the selected compound is indicated to inhibit the secretion of toxins by virulent bacteria.

To contact a candidate compound with FlhB_{C} or a paralog thereof, any technique known in the art can be used which enables the existence of the candidate compound and FlhB _{C} or a paralog thereof at the same location. The candidate compound can be contacted with FlhB_{C} in solid, in solution, or in atmosphere. The step of contacting can also be performed in silico, as described in detail herein below.

To analyze interaction of the candidate compound with or around a loop region of FlhB_{C} or a paralog thereof. In accordance with the present invention, any technique known in the art can be used which enables the determination of the interaction manner between the candidate compound and FlhB_{C} or a paralog thereof. Such technique includes, but not limited to, surface plasmon resonance such as Biacore, isothermal titration calorimetry (ITC), and fluorescence resonance energy transfer (FRET). The step of contacting can also be performed in silico, as described in detail herein below.

To select a compound that reduces flexibility of the loop region, or a linker that connects the transmembrane and cytoplasmic domains of FlhB, any technique known in the art can be used which enable the determination of the flexibility of FlhB_{C}. The MDS assay, secretion assay and motility assay disclosed in the Example can be used for this step.

For each step of the method of present invention, in silico technique known in the art can also be employed which uses the structural information of FlhB_{C} disclosed herein. For example, computer modeling can be performed using a docking program such as GRAM, DOCK, HOOK or AUTODOCK (Dunbrack, et al. (1997) Folding & Design 2:27-42). Alternatively, GRID (Molecular Discovery Ltd., UK) software package can be used to perform a chemical-probe approach. These techniques enable the simulation of compounds which have strong affinity with or around a loop region of FlhBc or a paralog thereof.

Alternatively, Fragment Based Lead Discovery (FBLD) method (Rees D. C., Congreve M., Murray C. W., Carr R. (2004). Nature Reviews Drug Discovery 3, 660-672) can be employed as in silico technique for the present invention. This method is the computational screening method using “fragment information” of commercially available compounds and structural information of interested protein. Primarily considered force in this method can be hydrogen bond. The detail of FBLD method will be explained in Examples herein below.

The candidate compound to be screened in the present invention can be any chemical entity. The candidate compound may include, but not limited to, an antibody, a fragment thereof, an aptamer and a small molecular compound.

A compound identified by the above screening method is also embraced in the present invention. The exemplary of selected compounds available commercially are listed in the following table:

| Com- | | |
|---|---|---|
| pound | Structure | Chemical name |
| 47 | 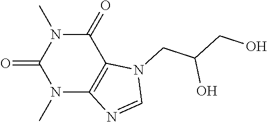 [SMILES: Cn1c(=O)c2c(ncn2CC(O)CO)n(C)c1=O] | 7-(2,3- dihydroxypropyl)- 1,3-dimethyl- 3,7-dihydro-1H- purine-2,6-dione |
| 64 | 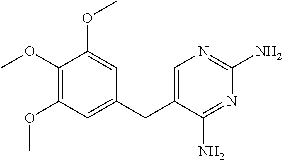 [SMILES: COc1cc(Cc2cnc(N)nc2N)cc(OC)c1OC] | 5-(3,4,5- Trimethoxybenzyl) pyrimidine- 2,4-diamine |

The identified compound can be formulated to a pharmaceutical composition.

The production of the pharmaceutical compositions can be effected in a manner which will be familiar to any person skilled in the art by bringing the identified compound and/or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavor-improving agents, salts for varying the osmotic pressure, buffer substances, solubilizers, colorants and masking agents and antioxidants come into consideration as pharmaceutical adjuvants.

The dosage can be vary within wide limits and will, of course, be fitted to the individual requirements in each particular case. In general, in the case of oral administration a daily dosage of about 10 to 1000 mg per person of the identified compound should be appropriate, although the above upper limit can be exceeded when necessary.

The pharmaceutical composition comprising the identified compound or a pharmaceutically acceptable salt thereof can be used for treating or preventing disorders caused by virulent bacteria using a needle type III secretion system. The disorders may include, but not limited to, stomach ache, diarrhea, nausea, vomit and convulsion. The pharmaceutical composition can be also used for inhibiting secretion of toxins into the host-cell cytoplasm by the virulent bacteria.

Treatment or prevention typically involves administering to a subject in need of treatment a pharmaceutical composition containing an effective dose of a compound identified in the screening method of the invention. In most cases this will be a human being, but treatment of agricultural animals. e.g., livestock and poultry, and companion animals, e.g., dogs, cats and horses, is expressly covered herein. The selection of the dosage or effective amount of a compound is that wichi has the desired outcome of preventing, reducing or reversing at least one sign or symptom of the disorder being treated.

The invention is described in greater detail by the following non-limiting examples.

### EXAMPLES

### Example 1

### Materials and Methods

1.1. Structure Determination

Details of purification of Salmonella and Aquifex FlhB_{C}, crystallization and data collection were described (Meshcheryakov, V. A. and Samatey, F. A. (2011). Acta Cryst. F67, 808-811; Meshcheryakov, V. A., Yoon, Y.-H. and Samatey, F. A. (2011). Acta Cryst. F67, 280-282). Both structures were solved by multiwavelength anomalous diffraction (MAD) using the program SHELXD (Sheldrick, G. M. (2008). Acta Cryst. A64, 112-122). Initial protein models were built automatically with Buccaneer (Cowtan, K. (2006). Acta Cryst. D62, 1002-1011) from the CCP4 package (Winn, M. D., Ballard, C. C., Cowtan, K. D., Dodson, E. J., Emsley, P., Evans, P. R., Keegan, R. M., Krissinel, E. B., Leslie, A. G. W., McCoy, A., McNicholas, S. J., Murshudov, G. N., Pannu, N. S., Potterton, E. A., Powell, H. R., Read, R. J., Vagin, A., Wilson, K. S. (2011) Acta Cryst. D67, 235-242). The models were refined through an iterative combination of refinement with Refmac5 (Murshudov, G. N., Skubak, P., Lebedev, A. A., Pannu, N. S., Steiner, R. A., Nicholls, R. A., Winn, M. D., Long, F., Vagin, A. A. (2011) Acta Cryst. D67, 355-367) and manual model building in COOT (Emsley, P., Lohkamp, B., Scott, W. G., Cowtan, K. (2010). Acta Cryst. D66, 486-501). In the case of Salmonella FlhB_{C}, TLS refinement was performed in the final stages with two TLS groups per FlhB_{C} molecule (residues 229-269 and 270-353) (Painter, J. and Merritt, E. A. (2006) Acta Cryst. D62, 439-450). Structural figures were made in PyMOL (http://www.pymol.org).

1.2. DNA Manipulation and Motility Assay

Mutations of S. typhimurium flhB carried by plasmid pMM26 (Non-Patent Literature 11) were done as previously described (Wang, W., Malcolm, B. A. (1999). BioTechniques, 26, 680-682). For the motility assay, freshly transformed Salmonella cells were inoculated as colonies directly into soft tryptone agar containing 0.35% (w/v) agar and incubated at 303 K.

1.3. Preparation of the Whole Cell and Culture Supernatant Fractions and Immunoblotting

Salmonella cells MKM50 (ΔflhB strain) (Non-Patent Literature 9) carrying an appropriate plasmid were incubated at 310 K in LB medium containing 100 μg ml⁻¹ of ampicillin until optical density OD600 reached 1.4-1.5. Aliquots of culture containing a constant amount of cells were centrifuged.

Cell pellets were suspended in an equal volume of SDS-loading buffer. Proteins in the culture supernatant were precipitated by 10% trichloroacetic acid and suspended in SDS-loading buffer. After SDS-PAGE, proteins were detected with anti-FlgE and anti-FliC antibodies using a WesternBreeze® chromogenic immunodetection kit (Invitrogen).

1.4. Molecular Dynamics Simulation

Molecular dynamics (MD) simulations were performed using the SCUBA (Simulation Codes for hUge Biomolecular Assembly) program package (Ishida, H., Higuchi, M., Yonetani, Y., Kano, T., Joti, Y., Kitao, A., Go, N. (2006). Annual Report of the Earth Simulator Center, 237-239). The AMBER ff99SB force-field (Hornak, V., Abel, R., Okur, A., Strockbine, B., Roitberg, A., Simmerling, C. (2006) Proteins: Structure, Function and Bioinformatics, 65, 712-725) was used for the protein. The simulated systems were solvated with the SPC/E water molecules (Berendsen, H. J. C., Grigera, J. R., Straatsma, T. P. (1987). J. Phys. Chem. 91, 6269-6271) with 100 mM KCl in the periodic boundary separated by at least 12 Å from the FlhB_{C} molecule in the initial stage. After energy minimization and 0.27 ns MD simulation to adjust the temperature and pressure of the system to 300 K and 1 atm with positional restraints, 40 ns MD simulation was performed without restraints in the canonical ensemble. The last 20 ns trajectory was used for the analysis. A shifted-force cutoff of real space non-bonded energy was made at 12 A and the particle-particle-particle-mesh (PPPM) method (Deserno, M., Holm, C. (1998). J. Chem. Phys. 109, 7678-7693) was employed for electrostatic energy calculation in Fourier space. Integration of the equation of motion was carried out using the multi-time step method XORESPA (Martyna, G. J., Tuckerman, M. E., Tobias, D. J., Klein, M. L. (1996). Mol. Phys. 87, 1117-1157) in the canonical ensemble. Integrations of fast (bond and angle), medium (torsion and real space non-bonded) and slow (Fourier space non-bonded) energy terms were performed every 0.5, 1.0 and 2.0 fs, respectively.

1.5. Accession Numbers

Atomic coordinates and structure factors are deposited in the PDB with accession codes 3B0Z and 3B1S for Salmonella and Aquifex FlhB_{C}, respectively. The structures reported here are explained in interactive 3D at http://Proteopedia.Org/w/Samatey.

### Results

### Example 2

### Flagellar FlhB_{C} Structure Description

Salmonella (Sal FlhB_{C}) and Aquifex (Aqu FlhB_{C}) FlhB_{C} structures were solved by multiwavelength anomalous diffraction (MAD) using selenomethionine derivatives (Meshcheryakov et al., 2011; Meshcheryakov and Samatey, 2011) (Table 1).

TABLE 1
X-ray data collection and refinement statistics. Values in parentheses indicate statistics for the highest resolution
shell. MAD data collection statistics for Salmonella FlhB_{C} was published in Meshcheryakov and Samatey, 2011.

| | Salmonella FlhB_{C} | | | | Aquifex FlhB_{C} | | | | | |
|---|---|---|---|---|---|---|---|---|---|---|
| | Native | | Native | | SeMet derivative | | | | | |
| Data collection | | | | | | | | | | |
| Space group | P4₂2₁2 | | C2 | | C2 | | | | | |
| Cell dimensions | | | | | | | | | | |
| a, b, c (Å) | 49.1, 49.1, 143.1 | | 114.6, 33.8, 122.4 | | 113.4, 33.6, 122.2 | | | | | |
| α, β, γ (°) | 90, 90, 90 | | 90, 107.8, 90 | | 90, 107.9, 90 | | | | | |
| Molecules/A.U.^{a} | 1 | | 3 | | Peak | | Inflection | | Remote | |
| Wavelength (Å) | 0.9 | | 0.9 | | 0.9791 | | 0.97936 | | 0.99508 | |
| Resolution (Å) | 40.45-2.45 | (2.58-2.45) | 47.76-2.55 | (2.69-2.55) | 50-3.0 | (3.16-3.0) | 50-3.0 | (3.16-3.0) | 50-3.0 | (3.16-3.0) |
| R_{merge}^{b} | 0.075 | (0.380) | 0.056 | (0.386) | 0.094 | (0.452) | 0.069 | (0.407) | 0.064 | (0.368) |
| I/σI | 16.2 | (5.7) | 12.5 | (3.4) | 7.6 | (2.4) | 9.9 | (3.0) | 10.5 | (3.2) |
| Completeness (%) | 98.8 | (100) | 99.3 | (100) | 100 | (100) | 100 | (100) | 100 | (100) |
| Redundancy | 7.7 | (7.9) | 3.7 | (3.8) | 3.6 | (3.7) | 3.7 | (3.7) | 3.7 | (3.7) |

| | | |
|---|---|---|
| Refinement | | |
| Resolution (Å) | 28.07-2.45 | 29.75-2.55 |
| R_{work}/R_{free} | 23.1/24.7 | 24.1/26.2 |
| No. atoms | | |
| Protein | 992 | 2707 |
| Ligand/ion | 4 | 0 |
| Water | 20 | 48 |
| Wilson plot B-factor | 79.3 | 83.7 |
| Average B-factor | | |
| Protein | 78.8 | 73.6 |
| Ligand/ion | 77.2 | N/A |
| Water | 39.3 | 61.2 |
| R.m.s deviations | | |
| Bond lengths (Å) | 0.021 | 0.019 |
| Bond angles (°) | 2.090 | 1.844 |
| Ramachandran plot (%) | | |
| Most favoured | 97.5 | 99.7 |
| Additionally allowed | 2.5 | 0.3 |
| Disallowed | 0 | 0 |

^{a}A.U. (asymmetric unit).
^{b}R_{merge} = ΣₕₖₗΣ [symbol] |I [symbol] (hkl) − <I(hkl)>|/ΣₕₖₗΣ [symbol] I [symbol] (hkl), where I [symbol] (hkl) is the intensity of the i-th measurement of reflection hkl and <I(hkl)> is the mean value of I [symbol] (hkl) for all i measurements. [symbol]
indicates data missing or illegible when filed

Sal FlhB_{C} and Aqu FlhB_{C} crystals belonged to different space groups, P42212 and C2, respectively. In the case of the Aqu FlhB_{C} crystal there were three protein molecules in the asymmetric unit. Three molecules in the asymmetric unit are very similar, with RMSD for pairwise superposition ranging 0.40-0.76 Å. Each molecule consisted of two polypeptide chains resulting from proteolytic cleavage after Asn263. For all molecules no electron density was seen for the residues 213-231 on N-terminus; depending on the molecule, from 2 to 6 residues on C-terminus was disordered.

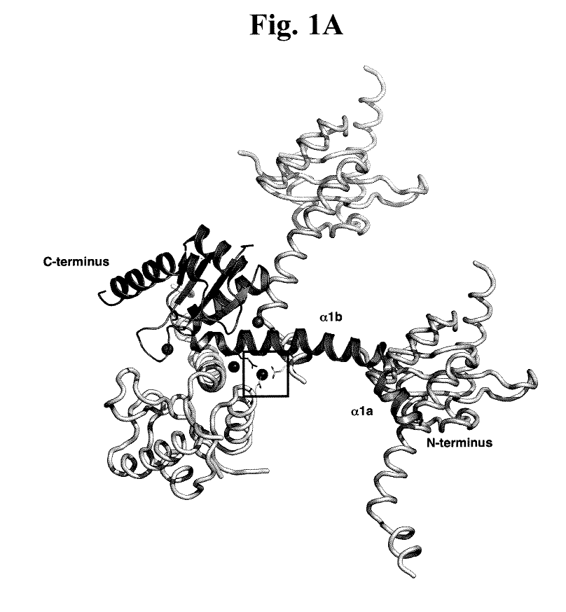

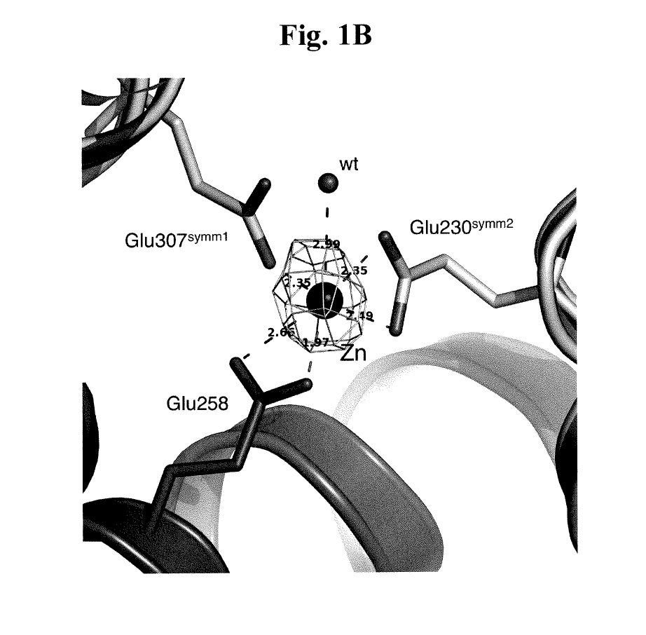

In the case of Sal FlhB_{C}, the final model comprised residues 229-353 out of 219-383 in the crystallized protein, with a cleavage after Asn269. No electron density was seen for the residues 219-228 and 354-383. The model of Salmonella FlhBc included two Zn and two Na ions (FIG. 1a). All these atoms mediated intermolecular interactions in the crystal lattice. Zn²⁺ was added to crystallization solution, and it was necessary to obtain well diffracting crystals. Analysis of the crystallographic packing showed that one of the zinc ions coordinated three glutamate residues from three symmetry-related SalFhB_{C} molecules: Glu230, Glu258, and Glu307 (FIG. 1b). This interaction makes N-terminal helix α1, one of the most flexible parts of Salmonella FlhB_{C} (see below), to be fixed between two symmetrical molecules.

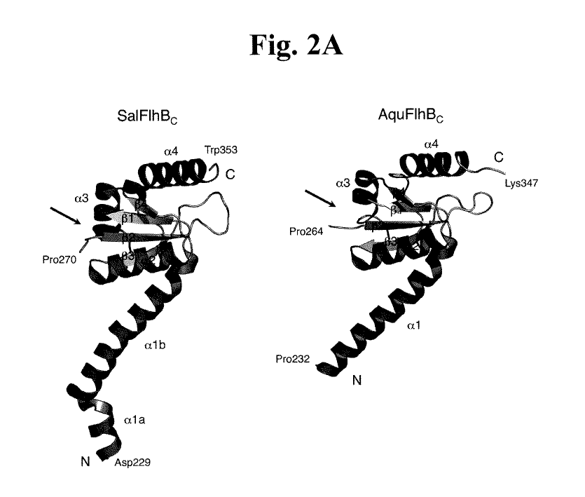

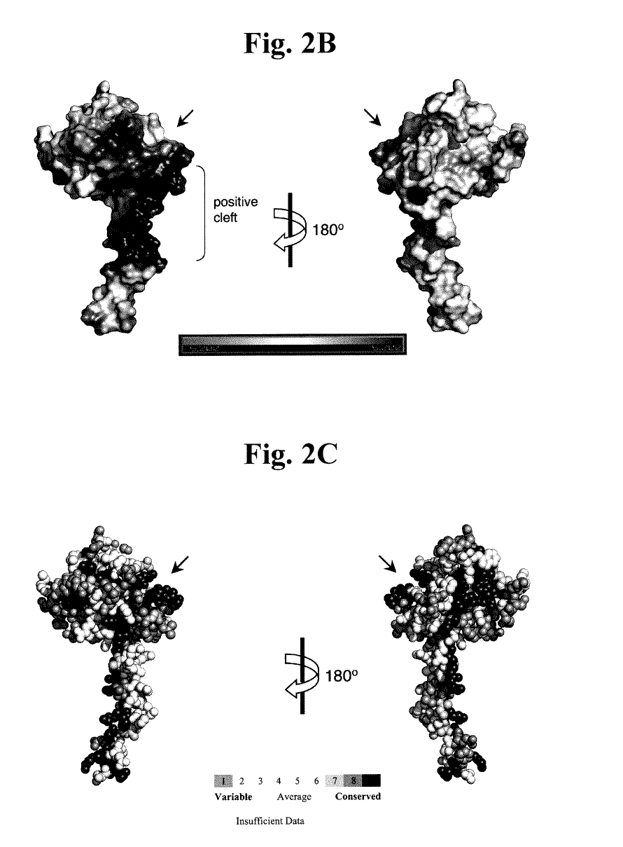

Both the Salmonella and Aquifex FlhB _{C} structures showed very similar folds with an RMSD of 1.03 A for 102 Cα atoms (FIG. 2a). Flagellar FlhB_{C} consisted of a globular domain composed of a four-stranded β-sheet, surrounded by four α-helices. The globular domain was preceded by a long Nterminal α-helix (α1) that connects the cytoplasmic globular part of FlhB to transmembrane domain. The α1 helix engaged in a crystal contact in both the Salmonella and Aquifex FlhB_{C} crystals, which may affect its orientation relative to the globular domain. However, these crystal contacts differed. In Sal FlhB_{C} crystal, α1 contacted primarily α1 and α2 of adjacent molecules, while in Aqu FlhB_{C} crystal, α1 contacted primarily α4 and the cleavage site between β1 and β2.

The major difference between Sal FlhB_{C} and Aqu FlhB_{C} is the N-terminal region. In the model of Sal FlhB_{C}, helix α1 was longer and had a kink at a very conserved residue Gly236. However, a longer helix with a kink was not excluded in Aqu FlhB_{C}, where highly conserved Gly230 occurred just 2 residues into the disordered segment 213-231 present in the crystallized protein but absent in the model. Although the kink may be due to the crystal packing, our data showed potential flexibility of the linker around this conserved glycine residue. The importance of such flexibility was previously shown for EscU, an FlhB paralog from the needle TTSS. Mutation of Gly229 (which corresponds to Gly236 of SalFlhB) to less flexible proline in EscU completely abolished secretion (Non-Patent Literature 10).

The conserved NPTH autocleavage site was exposed on a surface between strands β1 and β2. Both Salmonella and Aquifex FlhB_{C} showed different conformations of PTH region that suggested its flexibility. This was very different from the needle paralogs. In all known paralog structures, the PTH region has the same orientation, which is stabilized by the contacts with surrounding residues (Non-Patent Literature 10; Non-Patent Literature 18; Non-Patent Literature 19; Non-Patent Literature 20). It was difficult to say for the moment whether the greater flexibility of the PTH site in flagellar FlhB_{C} has any functional meaning. In Sal FlhB_{C}, the PTH region, together with adjacent residues in the globular domain and the C-terminal part of the linker α-helix, formed a positively charged cleft (FIG. 2b). A similar positive cleft was present also in Aqu FlhB_{C}. Such a cleft might be a potential recognition site for proteins secreted by the flagellar secretion system. The autocleavage of FlhB has been suggested to create an interaction site for other components of the type III secretion system (Non-Patent Literature 10; Non-Patent Literature 18). In particular, there is a model describing the binding of FliK to the cleaved NPTH loop of FlhB (Non-Patent Literature 21). However, the linker helix, which was one of the most conserved parts of FlhB protein (FIG. 2c), could also participate in the recognition of secreted proteins, since deletions or point mutations in this region of FlhB completely block secretion (Non-Patent Literature 9; Non-Patent Literature 10).

### Example 3

### Comparison with Needle Paralog Structures

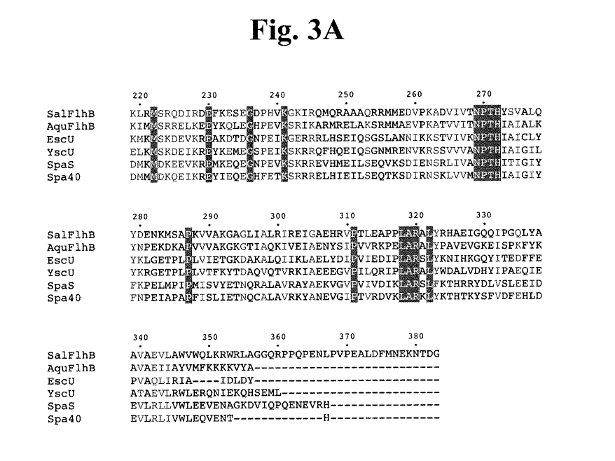

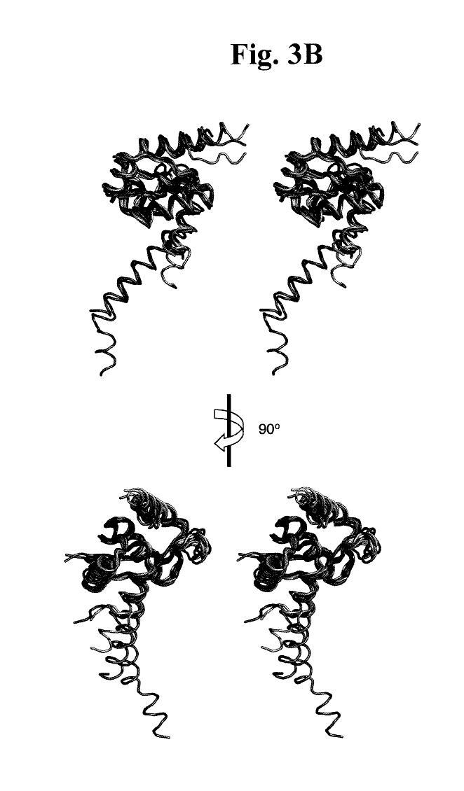

Despite a low sequence identity (FIG. 3a), the overall structure of flagellar FlhB_{C} was very similar to the structures of the paralogs from the needle secretion system: EscUC, SpaSC, YscUC, and Spa40C (FIG. 3b). The obvious difference between these proteins was the linker region between the N-terminal transmembrane domain and the globular cytoplasmic domain. All proteins showed a big difference in the conformation of their N-terminal parts, indicating flexibility of this region of the molecule. In our structures no electron density was observed for residues 219-228 of Sal FlhB_{C} and residues 213-231 of Aqu FlhB_{C}, which was consistent with flexibility of this part of FlhB_{C}.

However, the remaining of the residues of the linker formed a well-defined α-helix, which, in the case of Sal FlhB_{C}, was kinked at position Gly236. In contrast to the needle paralogs, it might be a general property of flagellar FlhB to have a more stable linker helix.

Proteins of the FlhB family exhibit significant variation in length mainly because of differences at the C-terminus. For instance, Salmonella FlhB is longer than Aquifex protein by 33 amino acids. However, these additional residues (residues 354-383) were not visible in the electron density map suggesting that they are unfolded. This region in SalFlhB was rich with proline residues making it unlikely to form any stable structure. The function of the elongated C-terminal part of FlhB is not known, but it is dispensable for motility (Non-Patent Literature 5). It apparently participates in the regulation of secretion because C-terminal truncation of Salmonella FlhB can partially suppress the phenotype of ΔfliK (Non-Patent Literature 5 and 7). However, it is unlikely to directly interact with FliK since the truncation has almost no effect in a wild-type fliK background (Non-Patent Literature 7).

### Example 4

### Effect of the Mutations of Residues 281-285 of Salmonella FlhB on TTSS Function

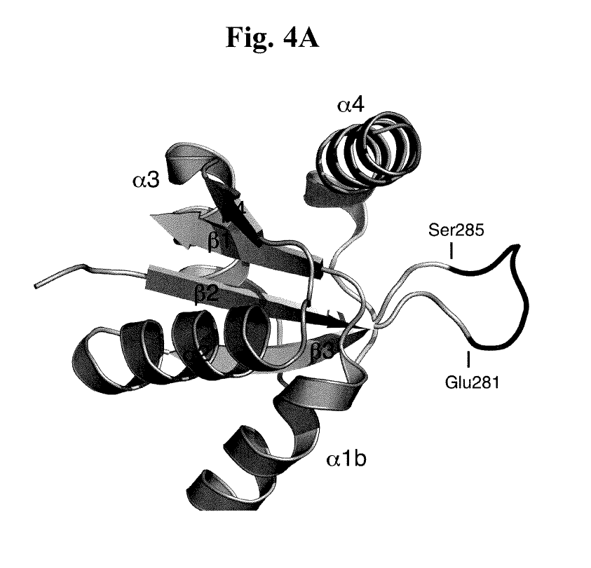

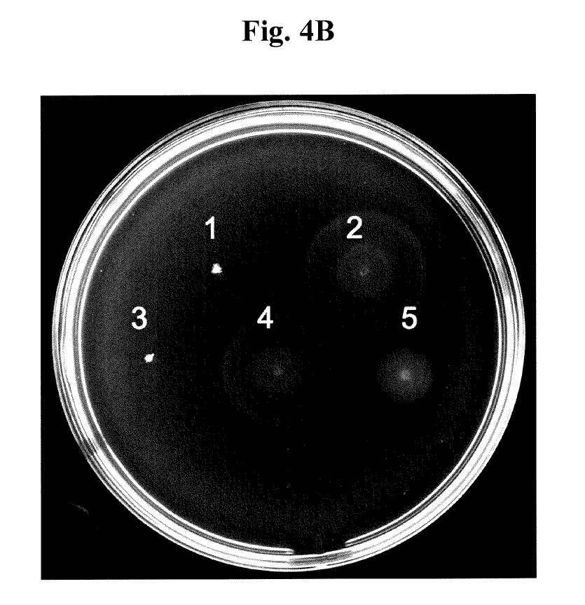

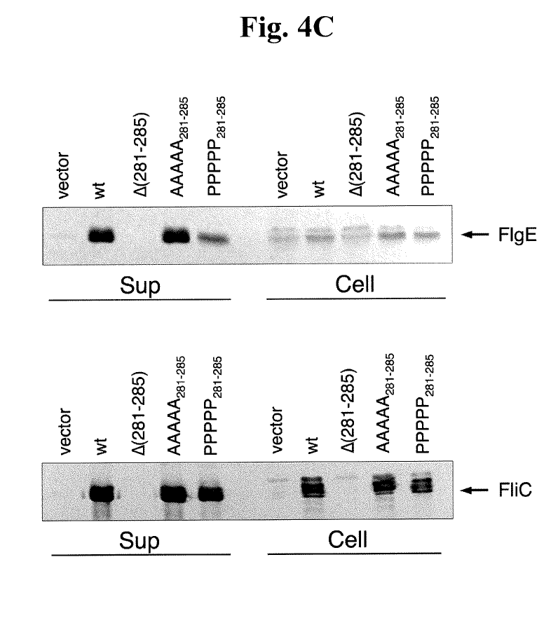

Two strands β2 and β3 were connected by a long flexible loop. This loop was not conserved within the FlhB family, although it is flanked by highly conserved residues, Tyr279 and Pro287 (in Salmonella numeration). The length of the loop, which was longer than necessary just for connecting two β-strands, made us to think that it might be of functional importance. To investigate this hypothesis three mutants of Salmonella FlhB were created. In the first mutant the loop residues 281-285 were deleted (FIG. 4a). In the second and third mutants residues 281-285 were substituted by Ala or Pro residues, respectively. Then swarming assays on soft agar plates were carried out to investigate whether the Salmonella cells, containing mutated FlhB, were still motile. It was observed that deletion of the loop completely abolished motility (FIG. 4b). At the same time substitution by Ala residues had no effect on motility, and Pro substitution decreased motility. To check whether these changes in motility were because of changes in export activity, secretion of hookprotein FlgE and filament protein FliC was analyzed by the flagellar secretion system containing mutated FlhB (FIG. 4c). It was observed that motility is correlated to the secretion of FlgE and FliC. In the case where the loop (281-285) of FlhB is deleted neither FlgE nor FliC were secreted, whereas proline substitution reduced secretion of both proteins. No difference in secretion was observed for the wild-type FlhB and the Ala substitution.

### Example 5

### Molecular Dynamic Simulation

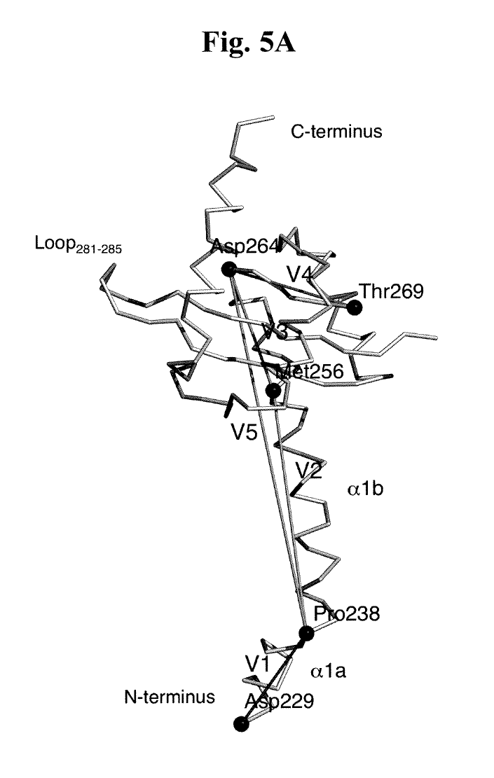

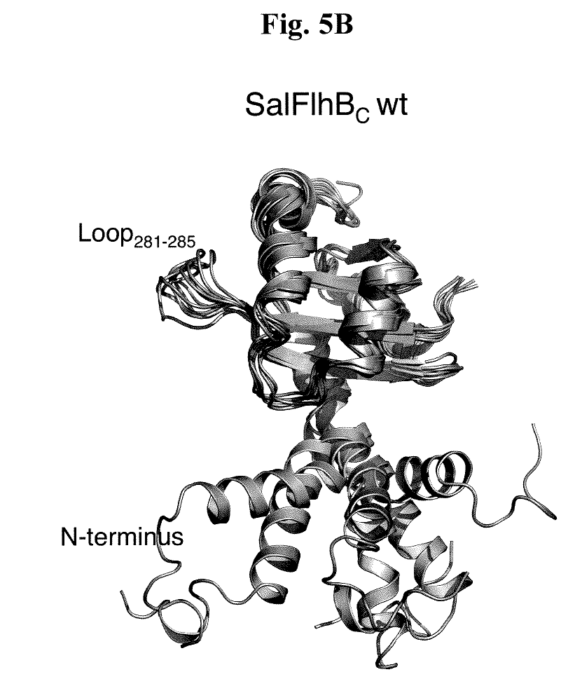

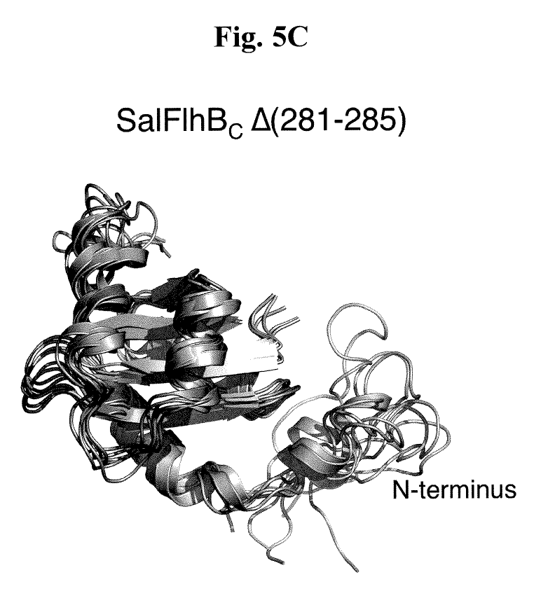

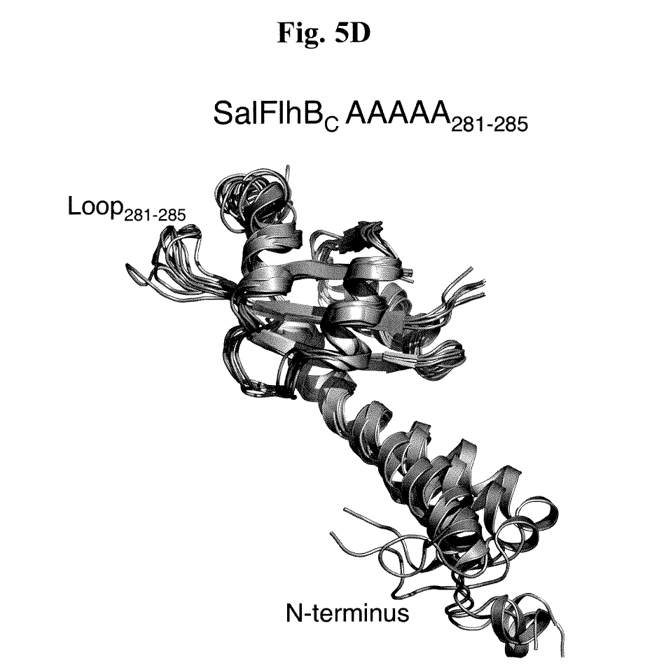

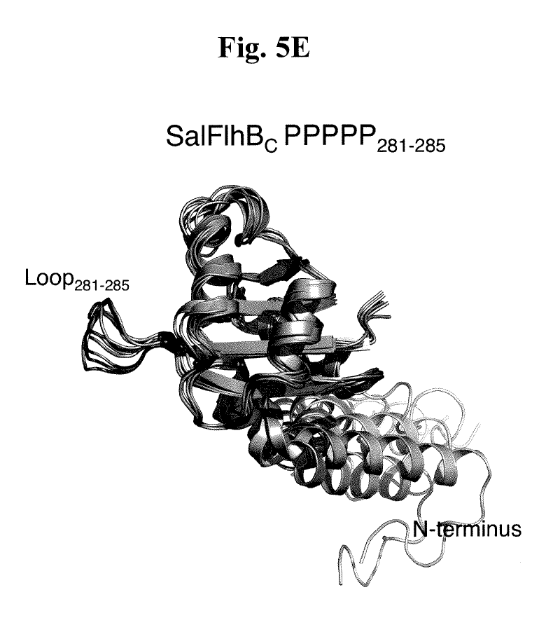

To further investigate the effect of the loop mutation on the FlhB_{C} molecule, MD simulation of the wild-type Sal FlhB_{C} and the Δ(281-285), AAAAA_{281-285,} and PPPPP₂₈₁₋₂₈₅ mutants was performed. During the MD, it was observed that the globular domain was relatively rigid in all the cases, while the N-terminal α-helix of the wild-type FlhB_{C} was very flexible and becomes less flexible in the mutants (FIG. 5b-e). In addition to the kink around Gly236-Pro238 (FIG. 2a), a significant kink was observed near Met256 during the MD. To characterize the flexibility of the N-terminal α-helix, a distance D, angles θ12, θ23, θ34, θ14 and torsion angles χ3 and χ5 were defined (see explanations in FIG. 5a and Table 2).

TABLE 2
Structure and fluctuation differences between wild-type Salmonella FlhB_{C} and its mutants during MD simulation
shown by key distance and angles defined by vectors V1-5 shown in FIG. 4a. D: length of V5. θ₁₂, θ₂₃, θ₃₄ and
θ₁₄: angles defined between V1 and V2, V2 and V3, V3 and V4, and V1 and V4, respectively. χ₃, and χ₅:
torsion angles defined by sets of three vectors V2-V3-V4 (torsion around V3) and V1-V5-V4 (around V5), respectively.
Averages and standard deviations over last 20 ns MD are shown, with negative values in boldface.

| Protein | D, Å | θ₁₂, ° | θ₂₃, ° | θ₃₄, ° | θ₁₄, ° | χ₃, ° | χ₅, ° |
|---|---|---|---|---|---|---|---|
| Salmonella FlhB_{C} wt | 37.2 ± 3.9 | 96.0 ± 21.9 | 53.8 ± 19.4 | 105.5 ± 2.2 | 77.1 ± 38.3 | 83.8 ± 23.5 | −103.8 ± 44.8 |
| Salmonella FlhB_{C} Δ(281-285) | 36.9 ± 0.7 | 133.2 ± 11.7 | 64.1 ± 5.2 | 108.2 ± 1.7 | 34.0 ± 9.5 | −23.1 ± 7.9 | 168.3 ± 21.0 |
| Salmonella FlhB_{C} AAAAA₂₈₁₋₂₈₅ | 38.1 ± 1.5 | 113.3 ± 34.6 | 45.7 ± 6.1 | 113.2 ± 1.8 | 47.9 ± 35.4 | 30.3 ± 11.6 | −175.5 ± 43.6 |
| Salmonella FlhB_{C} PPPPP₂₈₁₋₂₈₅ | 36.6 ± 3.1 | 69.6 ± 37.1 | 51.4 ± 12.2 | 112.9 ± 2.4 | 59.2 ± 26.9 | −77.3 ± 18.9 | −165.4 ± 43.1 |

A notable structural difference was demonstrated by torsion angle χ3, which determined the direction of the V2 region of the N-terminal α-helix relative to the globular domain. The χ3 value was positive for the wild-type FlhB_{C} and AAAAA₂₈₁₋₂₈₅ mutant but negative for the Δ(281-285) and PPPPP₂₈₁₋₂₈₅ mutants, which was consistent with the structural difference shown in FIG. 5b-e. Since the latter mutations reduced Salmonella motility, this structure change might have some functional effects. Another notable difference was seen as reduction in θ12, θ23, θ14, χ3 and χ5 fluctuations in the PPPPP281-285 mutant, indicating the significant structure change and flexibility loss of the N-terminal α-helix.

### Example 6

### in silico Screening of Candidate Compounds Interacting with Loop Region of FlhB_{C} from Salmonella typhimurium

Candidate compounds interacting with FlhBc were screened in silico by using Fragment Based Lead Discovery (FBLD) method (provided by PharmaDesign Inc., Japan). This method analyzes in silico the interaction between target protein and moieties of known chemical compound (herein after called as Scaffold).

6.1 Construction of Scaffold Database (Scaffold DB)

Information of Scaffold was generated by following procedure. First, 345,099 of commercially available chemical compounds (dealed by KISHIDA CHEMICAL Co., Ltd.) were selected in view of drug-like, structure to avoid, and molecular weight. Second, ligand information of Protein Data Bank (PDB) was obtained from Ligand Expo (http://ligand-expo.rcsb.org/) which organizes ligand information of PDB. Then, LIGPLOT interaction analysis (http://www.ebi.ac.uk/thornton-srv/software/LIGPLOT/) was performed to obtain hydrogen bond information between atoms of main/side chain of proteins and atoms of ligand, as protein-ligand interaction information. Using the information of the above commercially available chemical compounds as query and protein-ligand interaction information as database, OEChem TK (provided by OpenEye: http://www.eyesopen.com/oechem-tk/), which can screen substructure, was performed to obtain information of candidate compounds which can mimic the substructure of ligands. Then, Small Molecule Subgraph Detector (SMSD: http://www.ebi.ac.uk/thornton-srv/software/SMSD/) was used to perform superposing the information of candidate compound to PDB ligand. 889 compounds were finally extracted as Scaffold DB by comparing and examining the result of SMSD and the result of the above protein-ligand interaction information.

6.2 Determination of Scaffold which Recognize Amino Acids Around Loop Region Using Structural Information of FlhB_{C}

Among the amino acid sequence ENKMS of loop region of FlhB_{C} from Salmonella typhimurium, residues of ENKS were involved in hydrogen bond. Thus, the compounds which bond with these ENKS residues by hydrogen bonding were selected from Scaffold DB. The selected compounds were ranked in view of partition coefficient and solubility calculated by StarDrop (provided by Optibrium: http://www.optibrium.com/stardrop/).

Further, since amino acid sequence PEKDK of loop region of Aqu FlhB_{C} aeolicus includes the Asparagine residue which was oriented toward the inward side of the loop in the structural information of Aqu FlhB_{C}, the compounds which bond with Asparagine residues by hydrogen bonding were also selected from Scaffold DB. The selected compounds were ranked by docking simulation using ASEDock of MOE (provided by Chemical Computing Group: http://www.optibrium.com/stardrop/).

As a result, 237 compounds were finally selected.

### Example 7

### Validation of Inhibitory Activity of Screened Compounds

To evaluate the inhibitory activity of the above selected compounds on secretion of toxin, following assays were performed as described previously (Non-Patent Literature 22).

7.1. Bacterial Growth Assay

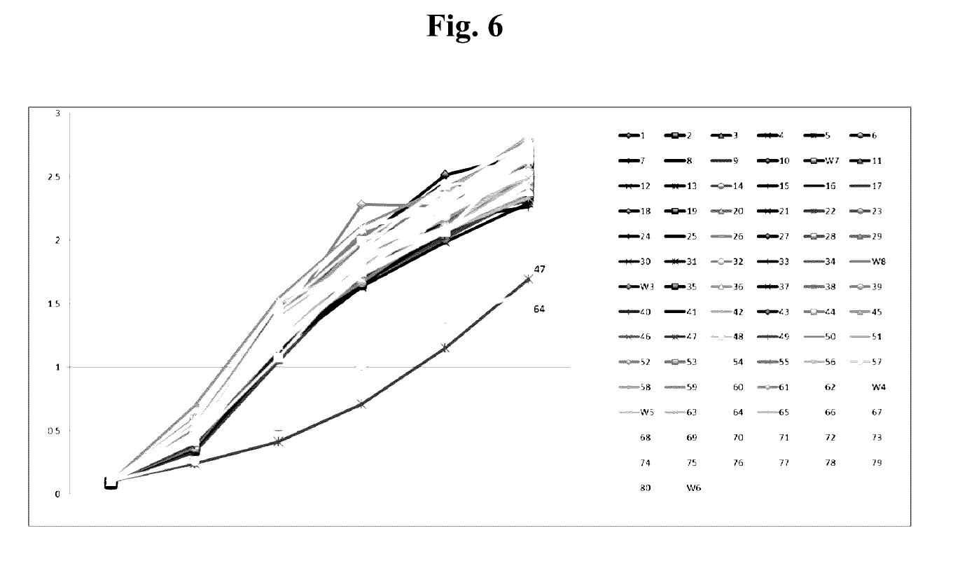

The effect of the candidate compounds on the viability of bacteria cultures were first tested before analyzing their effect on the flexibility of FlhB_{C}. 80 compounds out of the 237 compounds were purchased from KISHIDA CHEMICAL Co., Ltd according to the ranking. The Salmonella typhimurium strain SJW1103 was grown in LB medium at 303 K. Overnight cultures were diluted in Fresh LB medium to an optical density at 600 nm (OD600) of 0.1. Each of 5 μl of 100 mM candidate compounds solubilized in DMSO or DMSO alone as a control was added into 10 ml LB mediumin 50-ml volume polypropylene conicaltubes. For obtaining growth curves of the cell under the presence of each chemical, 0.2 ml of theculture volume were removed at every 2 h (2, 4, 6, 8 and 10 hr) and diluted with 1 ml of LB to measure absorbance at OD600. As a result, all 80 compounds did not lead to severe growth defects (FIG. 6). 2 compounds (compound 47 and compound 64) showed slight growth delay, but that slight delay was considered as not influential for the subsequent experiment.

7.2. Secretion Assay

Then, effects of the selected compounds on different type III secretion phenotypes were assessed. 1 ml of the cell suspensions of 8.0 hr culture in the above-growth assay were centrifuged at 13,200 rpm (16,100 g) for 10 min. The supernatant fractions (0.9 ml) were fractionated into new tubes and mixed with 100% trichloroacetic acid thoroughly. The fractions were kept on ice at least 1 h to precipitate secreted proteins. After centrifugation at 15,400 rpm for 30 min, the precipitated supernatant fractions were resuspended in 0.02 ml of 1 M Tris base and stored −30° C. until use. Samples for SDS-PAGE analysis were prepared by adding 5× SDS loading Buffer and then heat at 95° C. for 5 min. SDS-PAGE analyses were carried out at the condition of 200V for 30 min using premade gels purchased from Bio-rad. Electro-blotting onto PVDF membrane was performed with iBlot from Invitrogen and the membranes were developed with Chromogenic Immuno-detection Kit (Invitrogen) using a custom antibody raised against FlgD, a flagellar protein (from Prof. Keiichi Namba, Osaka University). Images were digitized as gray scale color with ChemDoc (Bio-rad). Kaleidoscope (Bio-rad) was used as a molecular marker.

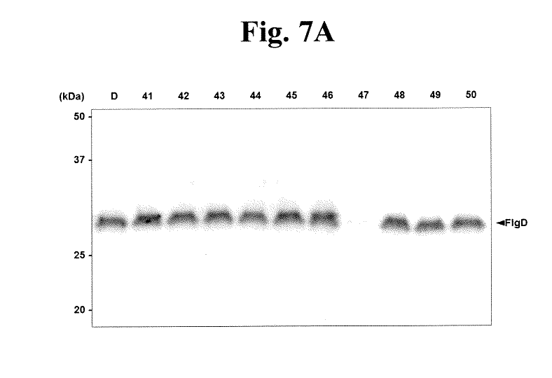

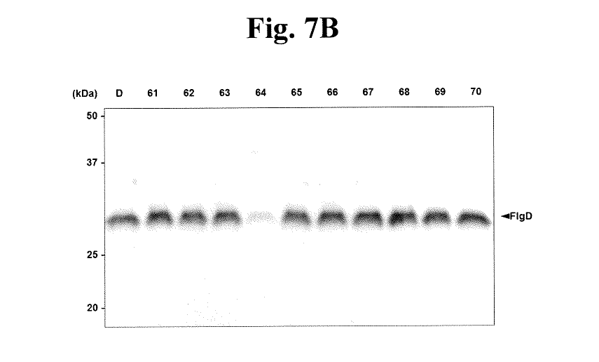

As a result, 7-(2,3-dihydroxypropyl)-1,3-dimethyl-3,7-dihydro-1H-purine-2,6-dione (compound 47: also known as dyphylline) and 5-(3,4,5-Trimethoxybenzyl)pyrimidine-2,4-diamine (compound 64: also known as Trimethoprim) were demonstrated to inhibit the secretion of FlgD, which is typical protein secreted by TTSS (Non-Patent Literature 11 and Non-Patent Literature 13) (FIG. 7a and FIG. 7b). This inhibitory activity of the compounds suggested the effects of compounds on inhibition of flexibility of FlhB_{C}.

What is claimed is:

1. A method of treating a disorder caused by virulent bacteria using a needle type III secretion system, the method comprising:
contacting the bacteria with a pharmaceutical composition; and
inhibiting the secretion of toxins by the virulent bacteria,
wherein the pharmaceutical composition comprises a compound selected from the group consisting of 7-(2,3-dihydroxypropyl)-1,3-dimethyl-3,7-dihydro-1H-purine-2,6-dione and 5-(3,4,5-Trimethoxybenzyl)pyrimidine-2,4-diamine, and a pharmaceutical adjuvant or a preservative.

2. A method for inhibiting secretion of toxins into the host-cell cytoplasm by virulent bacteria using a needle type III secretion system, the method comprising:
contacting the bacteria with a pharmaceutical composition; and
inhibiting the secretion of toxins by the virulent bacteria,
wherein the pharmaceutical composition comprises a compound selected from the group consisting of 7-(2,3-dihydroxypropyl)-1,3-dimethyl-3,7-dihydro-1H-purine-2,6-dione and 5-(3,4,5-Trimethoxybenzyl)pyrimidine-2,4-diamine, and a pharmaceutical adjuvant or a preservative.

3. A method for treating a disorder caused by virulent bacteria using a needle type III secretion system, comprising:
administering a pharmaceutically effective dose of a compound selected from the group consisting of 7-(2,3-dihydroxypropyl)-1,3-dimethyl-3,7-dihydro-1H-purine-2,6-dione and 5-(3,4,5-Trimethoxybenzyl)pyrimidine-2,4-diamine to a subject in need thereof for inhibiting secretion of toxins into the host-cell cytoplasm.

4. The method for treating a disorder according to claim 1, wherein the disorder is selected from the group consisting of stomach ache, diarrhea, nausea, vomit and convulsion.

5. The method for treating a disorder according to claim 3, wherein the disorder is selected from the group consisting of stomach ache, diarrhea, nausea, vomit and convulsion.

6. The method for treating a disorder according to claim 1, wherein the virulent bacteria is selected from the group consisting of Salmonella typhimurium, Aquifex aeolicus, Yersinia pestis, Shigella flexneri, enterohemorrhagic Escherichia coli, Pseudomonas aeruginosa, and Vibrio parahaemolyticus.

7. The method for treating a disorder according to claim 3, wherein the virulent bacteria is selected from the group consisting of Salmonella typhimurium, Aquifex aeolicus, Yersinia pestis, Shigella flexneri, enterohemorrhagic Escherichia coli, Pseudomonas aeruginosa, and Vibrio parahaemolyticus.
